(19) United States
(12) Patent Application Publication
(10) Pub. No.: US 2022/0403036 A1
(43) Pub. Date: Dec. 22, 2022

(54) P-SELECTIN INHIBITOR, COMPOSITIONS THEREOF AND METHODS OF USE THEREOF

(71) Applicant: THE JOHN'S HOPKINS UNIVERSITY, Baltimore, MD (US)

(72) Inventor: Charles J. Lowenstein, Baltimore, MD (US)

(21) Appl. No.: 17/842,580

(22) Filed: Jun. 16, 2022

Related U.S. Application Data
(60) Provisional application No. 63/212,056, filed on Jun. 17, 2021.

(51) Int. Cl. C07K 16/28; A61P 13/12; A61K 9/00

(52) U.S. Cl. CPC C07K 16/2854; A61P 13/12; A61K 9/0029; A61K 9/0014

(57) ABSTRACT

Provided herein are methods of uses of a P-selectin inhibitor alone or in combination with a Von Willebrand factor (VWF) inhibitor. The methods include the administration of a P-selectin inhibitor alone or in combination with a Von Willebrand factor (VWF) inhibitor for the treatment of COVID-19 associated endothelial injury and/or reducing the risk of thrombosis in a subject of need thereof.

### CROSS-REFERENCE TO RELATED APPLICATION(S)

This application claims benefit of priority under 35 U.S.C. § 119(e) of U.S. Provisional Patent Application Ser. No. 63/212,056, filed Jun. 17, 2021. The disclosure of the prior application is considered part of and is incorporated by reference in the disclosure of this application.

### STATEMENT REGARDING FEDERALLY SPONSERED GRANTS

This invention was made with government support under Grant No. R01 HL134894 and R61 HL141791. The government has certain rights in this invention.

### BACKGROUND OF THE INVENTION

### Field of the Invention

The present invention relates generally to P-selectin inhibitors and more specifically to the use of a P-selectin inhibitor alone or in combination with a Von Willebrand factor (VWF) inhibitor for the treatment of endothelial injury and/or reducing the risk of thrombosis.

### Background Information

Severe acute respiratory syndrome coronavirus 2 (SARS-CoV-2) is a highly infectious and pathogenic respiratory virus with a wide variety of respiratory, cardiovascular and systemic manifestations. Clinical studies suggest that endothelial cell injury and activation is part of the pathogenesis of COVID-19. Autopsies of patients who died from COVID-19 show systemic microvascular inflammation and microthrombi characteristic of the sequelae following activation of endothelial cells. Severe cases of COVID-19 are characterized by elevated levels of P-selectin and Von Willebrand factor (VWF), biomarkers of endothelial degranulation. However, the role of these vascular mediators in the pathogenesis of COVID-19 is unknown. Endothelial cell release of vasoactive compounds may play a central role in the pathogenesis of severe COVID-19.

### SUMMARY OF INVENTION

The present invention is based on the seminal discovery that a P-selectin inhibitor alone or in combination with a Von Willebrand factor (VWF) inhibitor is useful for the treatment of endothelial injury and/or reducing the risk of thrombosis in a subject.

In one embodiment, the present disclosure provides methods of reducing the risk of thrombosis in a subject in need thereof by administering to the subject a therapeutically effective amount of a P-selectin inhibitor, thereby reducing the risk of thrombosis in the subject. In one aspect, the P-selectin inhibitor is selected from an antibody, a nucleic acid molecule or a small molecule. In a further aspect, the P-selectin inhibitor is an antibody and the antibody is crizanlizumab. In other aspects, the P-selectin inhibitor is a nucleic acid molecule and the nucleic acid molecule is a short interfering nucleic acid (siNA), short interfering RNA (siRNA), double-stranded RNA (dsRNA), micro-RNA (miRNA), antagomir, aptamer, or a short hairpin RNA (shRNA) molecule. In further such aspects, the P-selectin inhibitor is a nucleic acid molecule, the nucleic acid molecule is an aptamer, and the aptamer is ARC5692. In a further aspect, the P-selectin inhibitor is a small molecule and the small molecule is PSI-697. In additional aspects, the subject with a risk of thrombosis is infected with SARS-CoV2.

In further aspects, the risk of thrombosis is associated with a microvascular, autoimmune and/or inflammatory condition. In some such aspects, the microvascular condition is microvascular thrombosis, thrombotic thrombocytopenic purpura (“TTP”), hemolytic uremic syndrome (“HUS”) or Disseminated Intravascular Coagulation (“DIC”). In other such aspects, the autoimmune condition is a type of vasculitis, rheumatoid arthritis (“RA”) or systemic lupus erythematosus (“SLE”). In additional such aspects, the inflammatory condition is organ inflammation, acute transplant rejection, acute kidney allograft rej ection, acute cardiac allograft rej ection, chronic transplant rejection, chronic cardiac allograft rejection, or chronic renal allograft rejection. In some aspects, the risk of thrombosis is associated the risk of acute brain injury during a procedure requiring a cardiac bypass.

In other aspects, administration of the P-selectin inhibitor leads to a reduction of soluble P-selectin in the subject. In some aspects, administration of the P-selectin inhibitor leads to a reduction of prothrombin fragment 1.2 and TAT complex in the subject. In additional such aspects, administration of the P-selectin inhibitor decreases the generation of thrombin.

In certain aspects, the P-selectin inhibitor is administered to the subject as a pharmaceutical composition in a therapeutically effective amount. In some such aspects, the pharmaceutical composition is administered via an enteral, topical or parenteral route of administration. In further such aspects, the route of administration is intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, oral, sublingual buccal, rectal, vaginal, nasal ocular administrations, or administration via infusion, inhalation, or nebulization.

In some aspects, the P-selectin inhibitor is administered via an intravenous route to the subject at a dosage of about 5 mg/kg. In certain such aspects, the P-selectin inhibitor is administered to the subject as a single intravenous infusion. In additional aspects, the P-selectin inhibitor is administered to the subject as an intravenous (IV) administration, e.g., infusion. For example, such IV administration is repeated in intervals, e.g., weekly, monthly, such as once a week, once every 2 weeks, once every three weeks, once every four weeks, once every five weeks, once every six weeks, once every seven weeks, once every eight weeks and the like.

In another embodiment, the present disclosure provides methods of treating a SARS-CoV2 associated endothelial injury in a subject by administering to the subject a P-selectin inhibitor and/or a VWF inhibitor, thereby treating the SARS-CoV2 associated endothelial injury. In certain such aspects, the P-selectin inhibitor is crizanlizumab, PSI-697 or ARC5692. In certain such aspects, the VWF inhibitor is integrilin or abciximab. In further aspects, the P-selectin inhibitor and/or the VWF inhibitor decrease vascular inflammation and vascular thrombosis. In other aspects, the P-selectin inhibitor and/or the VWF inhibitor inhibit endothelial-leukocyte interaction and endothelial-platelet interaction.

### BRIEF DESCRIPTION OF THE DRAWINGS

FIG. 1 is a diagram illustrating consolidated standard of reporting trials (CONSORT).

FIGS. 2A-2B illustrate the decrease of soluble P-selectin levels by crizanlizumab. FIG. 2A is a spaghetti plot illustrating P-selectin levels. FIG. 2B is a graph showing the comparison of soluble P-selectin levels in groups treated with crizanlizumab (“criz”) vs. placebo at baseline, day 3 or prior to discharge, and on the day of the discharge (n=22 placebo and n=20 crizanlizumab).

### DETAILED DESCRIPTION OF THE INVENTION

The present invention is based on the seminal discovery that a P-selectin inhibitor alone or in combination with a Von Willebrand factor (VWF) inhibitor is useful for the treatment of endothelial injury.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

All publications, patents, and patent applications mentioned in this specification are herein incorporated by reference to the same extent as if each individual publication, patent, or patent application was specifically and individually indicated to be incorporated by reference.

In one embodiment, the present disclosure provides methods of reducing the risk of thrombosis in a subject in need thereof by administering to the subject a therapeutically effective amount of a P-selectin inhibitor, thereby reducing the risk of thrombosis in the subject. In one aspect, the P-selectin inhibitor is selected from an antibody, a nucleic acid molecule or a small molecule. In a further aspect, the P-selectin inhibitor is an antibody and the antibody is crizanlizumab. In other aspects, the P-selectin inhibitor is a nucleic acid molecule and the nucleic acid molecule is a short interfering nucleic acid (siNA), short interfering RNA (siRNA), double-stranded RNA (dsRNA), micro-RNA (miRNA), antagomir, aptamer, or a short hairpin RNA (shRNA) molecule. In further such aspects, the P-selectin inhibitor is a nucleic acid molecule, the nucleic acid molecule is an aptamer, and the aptamer is ARC5692. In a further aspect, the P-selectin inhibitor is a small molecule and the small molecule is PSI-697. In additional aspects, the subject with a risk of thrombosis is infected with SARS-CoV2.

Severe acute respiratory syndrome coronavirus 2 (SARS-CoV-2) is a highly infectious and pathogenic respiratory virus with a wide variety of respiratory, cardiovascular and systemic manifestations. SARS-CoV-2 infection lead to the development of a disease, COVID-19. As used herein “COVID-19 associated endothelial injury” is meant to refer to any endothelial injury that is caused or induced (e.g., is the result of) by a SARS-CoV-2 infection. Non-limiting examples of COVID-19 associated endothelial injury include microvascular inflammation and microvascular thrombosis.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the COVID-19 associated endothelial injury). Such amount should be sufficient to reduce, limit or resolve microvascular inflammation and microvascular thrombosis. The effective amount can be determined as described herein.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the COVID-19 associated endothelial injury). Such amount should be sufficient to reduce, limit or resolve microvascular inflammation and microvascular thrombosis. The effective amount can be determined as described herein.

P-selectin is a type-1 transmembrane protein that in humans is encoded by the SELP gene. P-selectin functions as a cell adhesion molecule (CAM) on the surfaces of activated endothelial cells, which line the inner surface of blood vessels, and activated platelets. In resting endothelial cells, it is stored in granules called Weibel-Palade bodies. In resting platelets P-selectin is stored in a-granules. In response to inflammatory cytokines such as IL-4 and IL-13, P-selectin is translocated to the plasma membrane in endothelial cells. The extracellular region of P-selectin is composed of three different domains like other selectin types; a C-type lectin-like domain in the N-terminus, an EGF-like domain and a complement-binding protein-like domains (same as complement regulatory proteins: CRP) having short consensus repeats (about 60 amino acids- in length). The number of CRP repeats is the major feature differentiating the type of selectin in extracellular region. In human, P-selectin has nine repeats while E-selectin contains six and L-selectin has only two. P-selectin is anchored in transmembrane region that is followed by a short cytoplasmic tail region. The primary ligand for P-selectin is P-selectin glycoprotein ligand-1 (PSGL-1) which is expressed on almost all leukocytes, although P-selectin also binds to heparan sulfate and fucoidans. PSGL-1 is situated on various hematopoietic cells such as neutrophils, eosinophils, lymphocytes, and monocytes, in which it mediates tethering and adhesion of these cells. However, PSGL-1 is not specific for P-selectin, as it can also function as a ligand for both E- and L-selectin.

P-selectin plays an essential role in the initial recruitment of leukocytes (white blood cells) to the site of injury during inflammation. When endothelial cells are activated by molecules such as histamine or thrombin during inflammation, P-selectin moves from an internal cell location to the endothelial cell surface. Thrombin is one trigger which can stimulate endothelial-cell release of P-selectin and recent studies suggest an additional Ca2+-independent pathway involved in the release of P-selectin. Ligands for P-selectin on eosinophils and neutrophils are similar sialylated, protease-sensitive, endo-beta-galactosidase-resistant structures, clearly different than those reported for E-selectin, and suggest disparate roles for P-selectin and E-selectin during recruitment during inflammatory responses. P-selectin is also very important in the recruitment and aggregation of platelets at areas of vascular injury. In a resting platelet, P-selectin is located on the inner wall of α-granules. Platelet activation (through agonists such as thrombin, Type II collagen and ADP) results in “membrane flipping” where the platelet releases α- and dense granules and the inner walls of the granules are exposed on the outside of the cell. The P-selectin then promotes platelet aggregation through platelet-fibrin and platelet-platelet binding.

As used herein “P-selectin inhibitor” includes any molecule that can inhibit P-selectin function or interaction with its ligands. The term “molecule” includes, but is not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines. Non-limiting examples of polynucleotides include short interfering nucleic acid (siNA), antisense, enzymatic nucleic acid molecules, 2′,5′-oligoadenylate, triplex forming oligonucleotides, aptamers, and decoys. Biologically active molecules include antibodies (e.g., monoclonal, chimeric, humanized etc.), cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, allozymes, aptamers, decoys and analogs thereof, and small nucleic acid molecules, such as short interfering nucleic acid (siNA), short interfering RNA (siRNA), double-stranded RNA (dsRNA), micro-RNA (miRNA), antagomirs, and short hairpin RNA (shRNA) molecules.

In some aspects, the administration of the P-selectin inhibitor can be in combination with one or more additional therapeutic agents. The phrases “combination therapy”, “combined with” and the like refer to the use of more than one medication or treatment simultaneously to increase the response. The P-selectin inhibitor might for example be used in combination with other drugs or treatment in use to treat endothelial injury. Specifically, the administration of a P-selectin inhibitor to a subject can be in combination with a Von Willebrand factor (VWF) inhibitor. Such therapies can be administered prior to, simultaneously with, or following administration of the composition of the present invention.

In one aspect, the method further includes the administration of a Von Willebrand factor (VWF) inhibitor.

von Willebrand factor (VWF) is a blood glycoprotein involved in hemostasis. It is deficient and/or defective in von Willebrand disease and is involved in many other diseases, including thrombotic thrombocytopenic purpura, Heyde's syndrome, and possibly hemolytic-uremic syndrome. Increased plasma levels in many cardiovascular, neoplastic, and connective tissue diseases are presumed to arise from adverse changes to the endothelium and may predict an increased risk of thrombosis. Von Willebrand Factor's primary function is to bind to other proteins, in particular factor VIII and glycoprotein IIbIIIa. VWF plays several roles in hemostasis and thrombosis, including delivering factor VIII, mediating platelet adherence to the damaged vessel wall, and mediating platelet aggregation to other platelets. VWF is not an enzyme and, thus, has no catalytic activity.

One of the most important VWF ligands is factor VIII. Factor VIII is bound to VWF while inactive in circulation; factor VIII degrades rapidly when not bound to VWF. Factor VIII is released from VWF by the action of thrombin. In the absence of VWF, factor VIII has a half-life of 1-2 hours; when carried by intact VWF, factor VIII has a half-life of 8-12 hours. VWF binds to collagen, e.g., when collagen is exposed beneath endothelial cells due to damage occurring to the blood vessel. Endothelium also releases VWF which forms additional links between the platelets' glycoprotein Ib/IX/V and the collagen fibrils. VWF binds to platelet gpIb when it forms a complex with gpIX and gpV; this binding occurs under all circumstances but is most efficient under high shear stress (i.e., rapid blood flow in narrow blood vessels, see below). VWF binds to other platelet receptors when they are activated, e.g., by thrombin (i.e., when coagulation has been stimulated). VWF plays a major role in blood coagulation. Therefore, VWF deficiency or dysfunction (von Willebrand disease) leads to a bleeding tendency, which is most apparent in tissues having high blood flow shear in narrow vessels.

As used herein “VWF inhibitor” includes any molecule that can inhibit VWF function or interaction with its ligands. For example, a VWF inhibitor is a molecule that inhibits interaction of VWF with platelet.

In one aspect, the P-selectin inhibitor is an antibody or a small molecule.

As used herein the terms “antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. “Antibody,” as used herein, encompasses any polypeptide comprising an antigen-binding site regardless of the source, species of origin, method of production, and characteristics. Antibodies include natural or artificial, mono- or polyvalent antibodies including, but not limited to, polyclonal, monoclonal, multispecific, human, humanized or chimeric antibodies, single chain antibodies, and antibody fragments. “Antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]).

The term “antibody,” as used herein, refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed αε of two identical light (L) chains and two identical heavy (H) chains. The light chains from any vertebrate species can be assigned to one of two clearly distinct types, called kappa (κ) and lamdba (λ), based on the amino acid sequences of their constant domain. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

Experimentally, antibodies can be cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

In some aspects, the P-selectin inhibitor is crizanlizumab, PSI-697 or ARC5692.

In another aspect, the VWF inhibitor is a GPIIb/IIIA inhibitor.

Glycoprotein IIb/IIIa (GPIIb/IIIa, or integrin αIIbβ) is an integrin complex found on platelets. It is a receptor for fibrinogen and von Willebrand factor and aids platelet activation. The complex is formed via calcium-dependent association of gpIIb and gpIIIa, a required step in normal platelet aggregation and endothelial adherence. Platelet activation by ADP (blocked by clopidogrel) leads to the aforementioned conformational change in platelet GPIIb/IIIa receptors that induces binding to fibrinogen. The GPIIb/IIIa receptor is a target of several drugs including abciximab, eptifibatide, and tirofiban. As used herein, the term “glycoprotein IIb/IIIa inhibitors”, or “GPIIb/IIIa inhibitors” refers to a class of antiplatelet agents, that are frequently used during percutaneous coronary intervention (angioplasty with or without intracoronary stent placement). They work by preventing platelet aggregation and thrombus formation and do so by inhibition of the GPIIb/IIIa receptor on the surface of the platelets. They may also be used to treat acute coronary syndromes, without percutaneous coronary intervention, depending on TIMI risk. Non-limiting examples of GPIIb/IIIa inhibitors include abciximab (abcixifiban), eptifibatide (integrilin), tirofiban (aggrastat), roxifiban and orbofiban.

In some aspects, the VWF inhibitor is integrilin or abciximab.

In one aspect, the P-selectin inhibitor and/or the VWF inhibitor decrease vascular inflammation and vascular thrombosis.

In another aspect, the P-selectin inhibitor and/or the VWF inhibitor inhibit endothelial-leukocyte interactions and endothelial-platelet interaction.

Presented below are examples discussing P-selectin inhibitors contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Methods

### Trial Design and Oversight

The CRIzanlizumab for treatIng COVID-19 vAscuLopathy (CRITICAL) trial was a randomized, double-blind, placebo-controlled pilot trial. A steering committee designed and oversaw the conduct of the trial and data analysis. Data were collected, managed, and analyzed by the investigators. SOCAR Research was responsible for the generation and implementation of the randomization process and for data management using the eSOCDATTM web-based System and the data coordinating center at Brigham and Women's Hospital was responsible for analyzing the data. The funder (Novartis AG) provided an institutional grant to The Johns Hopkins University School of Medicine as well as study drug but had no role in the design, conduct or analysis of the trial data. The first draft of the manuscript was prepared by the last author who had complete access to the data. All the authors made the decision to submit the manuscript for publication and vouch for the accuracy and completeness of the data and for the fidelity of the trial to the protocol. This clinical trial was conducted at three hospitals within The Johns Hopkins Health System in Maryland, USA. The Johns Hopkins University School of Medicine Institutional Review Board (IRB) approved the protocol for the trial of an independent Data Safety Monitoring Board (DSMB). An independent data and safety monitoring board (DSMB) monitored trial conduct and patient safety and reviewed unblinded data during the course of the trial (Table 10).

### Trial Patients

Eligibility requirements at screening included: age of 18 years or older, symptoms of acute respiratory infection, SARS-CoV-2 infection within the past 10 days of randomization as documented by a positive SARS-CoV-2 laboratory test (reverse transcriptase polymerase chain reaction), hospitalization, need for supplemental oxygen or a peripheral capillary oxygenation saturation (SpO2)<94% on room air, radiographic evidence of pulmonary infiltrates, and elevated D-dimer>0.49 mg/L FEU. Exclusion criteria included mechanical ventilation, INR>3, or aPTT>60 seconds (Table 9). All patients provided written informed consent.

### Trial Procedures

The trial consisted of a screening period followed by a double-blind treatment period. After the screening period, patients were randomly assigned in a 1:1 ratio to receive double-blind treatment with one intravenous dose of crizanlizumab 5.0 mg/kg or placebo. Randomization was performed using a secure, central, interactive, web-based response system (eSOCDATTM). Crizanlizumab or saline placebo was prepared by the site pharmacist who was unblinded; after crizanlizumab or placebo was prepared, the study treatment was blinded by using darkened bags. After randomization, patients were evaluated at days 3, 7, 14 if hospitalized, as well as day of discharge. Collection of blood samples stopped at hospital discharge. Patients were contacted 30 days after discharge by follow-up telephone call.

### Trial Outcomes

The primary outcome was the difference between treatment groups in levels of soluble P-selectin at day 3 after randomization or hospital discharge day, whichever occurred earlier. Secondary outcomes included changes between the treatment groups at days 7 and 14 for levels of soluble P-selectin, and at days 3, 7, and 14 for levels of D-dimer, VWF, and CRP (provided study participants remained in the hospital). No blood collections were obtained after discharge. Additional secondary outcomes also included changes between groups in the World Health Organization (WHO) Ordinal Scale for COVID-19 Trials, the time to hospital discharge, and the safety of crizanlizumab. Exploratory endpoints included changes between the group receiving crizanlizumab and the group receiving placebo at days 3, 7, and 14 in levels of: fibrinogen, IL-6, TNF-alpha, Factor VIII, soluble ICAM-1, soluble VCAM-1, CCL2, troponin isoform T, and NTpro-BNP (Table 10). Exploratory endpoints included time to the following outcomes: resolution of fever, liberation from supplemental oxygen, mechanical ventilation, hospital death, arterial or venous vascular event, ischemic stroke, myocardial infarction and hospital discharge. Additional biomarker assays that were not pre-specified endpoints were added post-hoc, including prothrombin fragment 1.2, thrombin-anti-thrombin complex, plasmin-anti-plasmin complex, interleukin-8, and iterleukin-10.

### Laboratory Assay of Primary Endpoint

P-selectin levels were measured in plasma samples using an enzyme-linked immunosorbent assay (Human P-Selectin Quantikine ELISA Kit, DPSE00, R&D Systems) according to the manufacturer's instructions. This assay has a detection range between 1.2 and 50 ng/mL. Undetectable samples were assigned a value of the lower limit of detection/2 (0.605 ng/mL). All subjects in the placebo group and the crizanlizumab group had detectable P-selectin levels on day 0. All 20 subjects in the placebo group had detectable P-selectin levels on day 3, but 3 of the 22 subjects in the crizanlizumab group had P-selectin levels below the limit of detection on day 3.

### Statistical Analyses

The pre-specified primary, secondary, and exploratory analyses were based on a modified intention-to-treat (mITT) population that consisted of all randomized patients who had a valid baseline P-selectin value prior to study drug administration and for whom at least one post-baseline P-selectin value was available on or prior to day 3. Adverse events were assessed in all randomized patients for whom study treatment was started.

The primary endpoint was summarized for each treatment group with descriptive statistics, including mean, standard deviation, median, and interquartile range as appropriate. Evaluation of the primary endpoint was assessed in an analysis of covariance, with baseline soluble P-selectin and treatment group as covariates. A soluble P-selectin greater than the assay maximum measurement was imputed as 150% of the upper limit of assessment.

The pre-specified primary, secondary, and exploratory analyses were based on a modified intention-to-treat (mITT) population that consisted of all randomized patients who had a valid baseline P-selectin value prior to study drug administration and for whom at least one post-baseline P-selectin value was available on or prior to day 3. Adverse events were assessed in all randomized patients for whom study treatment was started. The primary endpoint was summarized for each treatment group with descriptive statistics, including mean, standard deviation, median, and interquartile range as appropriate. Evaluation of the primary endpoint was assessed in an analysis of covariance, with baseline soluble P-selectin and treatment group as covariates. A soluble P-selectin greater than the assay maximum measurement was imputed as 150% of the upper limit of assessment.

Characteristics between treatment groups were compared using t-tests for normally distributed continuous variables or Wilcoxon rank-sum tests for non-normally distributed continuous variables. Categorical variables were compared using Chi-Square or Fisher's exact tests, as appropriate. A p-value of <0.05 was considered significant. Secondary and exploratory endpoints were not adjusted for multiplicity.

Based on a trial design with two-sided a=0.05 and assuming a standard deviation (SD) of 30 ng/ml and a 10% drop out rate, a sample size of 40 patients (20 per treatment arm) was determined to provide 83% power to detect a between-group difference of 30 ng/ml and 93% power to detect a between-group difference of 35 ng/ml. All analyses were conducted using Stata, version 16 (StataCorp).

### Example 2

### Results and Discussion

### Enrollment, Randomization, and Follow-Up

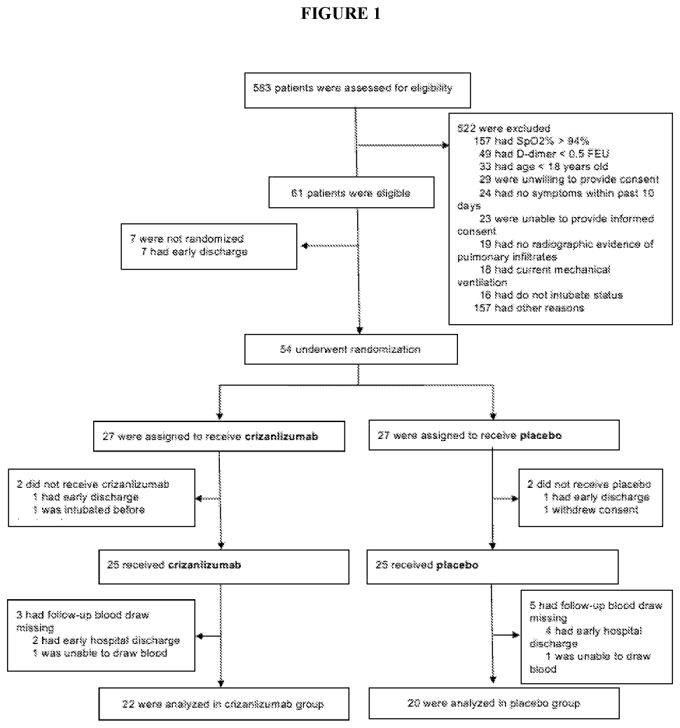

583 patients were screened at 3 hospitals within the Johns Hopkins Health System (FIG. 1). A total of 54 patients who fulfilled study entry criteria were randomized to receive crizanlizumab (n=27) or placebo (n=27). Out of the 27 assigned to receive crizanlizumab, 2 did not receive crizanlizumab because of early discharge or early intubation; out of the 25 who received crizanlizumab, 3 had follow-up blood draws missing due to early hospital discharge, and 22 patients were analyzed in the crizanlizumab group. Out of the 27 assigned to receive placebo, 2 did not receive placebo because of early discharge or withdrawn consent; out of the 25 who received placebo, 5 had follow-up blood draws missing due to early hospital discharge, and 20 patients were analyzed in the placebo group. Therefore, a total of 22 patients assigned to crizanlizumab and 20 patients assigned to placebo had blood available for biomarker analyses and were included in the primary analysis. At the end of the trial, outcomes were known for all patients.

The baseline clinical characteristics of the patients between the treatment groups were moderately well balanced for a small sample size, except for a history of coronary artery disease and race (Table 1). There were no significant differences between groups for baseline levels of blood biomarkers (Table 2).

### Outcomes

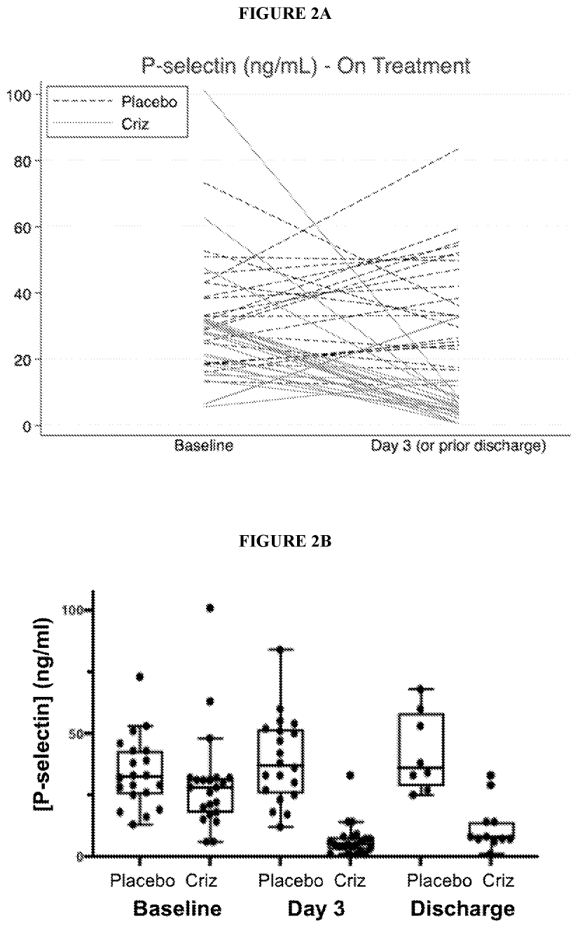

Baseline levels of P-selectin were similar between patient groups before receiving crizanlizumab (30±20 ng/mL) and placebo (34±15 ng/mL). The mean±SD changes from baseline to day 3 or before discharge were −23±23 ng/mL and [symbol] 5±18 ng/mL. In baseline-adjusted models, crizanlizumab reduced P-selectin levels by 32 ng/mL (95% CI: 24-41; P<0.001) by day 3 or before discharge (89% reduction from baseline), 38 ng/mL: (95% CI: 28-48; P<0.001) by day 7 (84% reduction from baseline), and 35 ng/mL: (95% CI: 19-51; P<0.001) by day 14 (80% reduction from baseline) (See FIG. 2 and Table 3).

Crizanlizumab increased D-dimer levels by 77% (CI 6%, 194%; P=0.03) on day 3 after treatment; D-dimer levels were not significantly different between groups on day 7 and on day of discharge after day 3. Crizanlizumab had no significant effect on other secondary biomarker endpoints, including levels of IL-6, TNF-alpha, VWF, and CRP (Table 4).

No difference was observed in clinical outcomes between treatment groups, including time to hospital discharge, or clinical status measured by the WHO ordinal scale for COVID-19 trials (Table 5, Table 6). There were no events for the endpoints of duration of mechanical ventilation, time to vascular event, stroke or myocardial infarction.

Several post-hoc analyses were performed to better understand the effect of crizanlizumab upon markers of thrombosis and lysis. Crizanlizumab had no effect on a wide variety of cytokine and chemokine biomarkers, with the exception of IL-8 and IL-10 which were both significantly increased on day 3 post randomization (Table 7). To assess the effect of crizanlizumab upon thrombin activation, we measured prothrombin fragment 1.2 and thrombin-anti-thrombin (TAT) complex. Crizanlizumab decreased prothrombin fragment 1.2 to 2.3 ng/ml (95% CI: 1.2-3.8) compared to placebo 3.6 ng/ml (95% CI 0.6-6.1) with P=0.007 on day 3, and non-significantly decreased TAT complex 4.3 ng/ml (CI 3.0, 7.8) compared to placebo 8.4 ng/ml (CI 4.8, 9.98) with P=0.08 on day 3 (Table 7). Crizanlizumab had no effect on PAP complex 312 ng/ml (CI 205, 737) compared to placebo 345 ng/ml (180, 788) with P =0.93 on day 3 (Table 7).

### Safety

After randomization there were 6 serious and non-serious adverse events in the placebo treated group of 25 patients and 7 serious and non-serious adverse events in the crizanlizumab treated group of 25 patients, including 1 serious adverse event (multi-organ failure) in the placebo group and 0 serious adverse events in the crizanlizumab group (Table 8). Non-serious adverse events included headache, change in mental status, chest pain, urinary tract infection, and diarrhea.

### Discussion

It was found that crizanlizumab markedly reduced soluble P-selectin levels in patients with moderate COVID-19 without affecting several cytokines. Crizanlizumab increased D-dimer levels and decreased prothrombin fragment 1.2. Adverse events were similar between the crizanlizumab and placebo groups.

Crizanlizumab, a soluble P-selectin inhibitor, has previously been shown to reduce the rate of sickle cell-related vaso-occlusive crises in patients with sickle-cell disease. Elevation of P-selectin and VWF, both contained in endothelial granules, has been reported in COVID-19 and associated with worse outcomes. Therefore, it was hypothesized that blocking P-selectin with crizanlizumab would have beneficial downstream effects on inflammatory markers and markers of thrombosis by blocking leukocyte rolling, an early step in leukocyte trafficking that is mediated by P-selectin. Nevertheless, we observed no compelling differences between treatment groups in markers of systemic inflammation such as CRP, IL-6, TNF-alpha, or IFN-gamma. Crizanlizumab also did not affect markers of vascular inflammation released from endothelial cells, such as VCAM-1 or CCL2 (also called MCP1). Crizanlizumab did increase levels of IL-10, an anti-inflammatory cytokine without suppressing targets of IL-10 such as TNF-alpha, IL-1β, and IFN-gamma, so the significance of increased IL-10 levels is unclear. Crizanlizumab also increased levels of IL-8, a pro-inflammatory cytokine that recruits neutrophils. It is unclear how inhibition of P-selectin leads to changes in IL-8 and IL-10. The finding that markers of inflammation, particularly IL-6 and CRP, were not reduced by crizanlizumab suggests that P-selectin probably does not mediate the inflammatory response in COVID-19. The possibility that endothelial granule release may be secondary to inflammatory processes cannot be excluded.

It was found that crizanlizumab was associated with an increase in D-dimer which has been associated with the severity of COVID-19. D-dimer, a fibrin degradation product, is typically elevated in the setting of thrombosis and concurrent thrombolysis. Nevertheless, no evidence of increased macro-thrombosis or worsening of clinical status with crizanlizumab was observed. It is well known that D-dimer can also be elevated by therapies that stimulate thrombolysis. It was found that crizanlizumab reduced both prothrombin fragment 1.2 and TAT complex, biomarkers of thrombin activation. In prior studies of patients with COVID-19, elevation of prothrombin fragment 1.2 was highly correlated with D-dimer. Thus, the rise in D-dimer observed in association with a decrease in prothrombin fragment 1.2 suggests that crizanlizumab may stimulate fibrinolysis and reduce thrombin activation in COVID-19. These findings raise the possibility that crizanlizumab might be beneficial by reducing microvascular thrombosis, which is common in severe disease.

While no effect on clinical outcomes in patients treated with crizanlizumab were observed, the patients enrolled in this trial was small in number and relatively healthy. There were no deaths during the study period. Average oxygen saturation levels in enrolled patients were between 87%-93% for patients on room air and between 94%-95% for patients on supplemental oxygen, CRP levels were between 5.8-7.6 mg/dL at baseline, and the clinical course in the majority of patients was relatively benign. It is possible that the benefits of crizanlizumab may be greater in a higher risk population with more severe vascular injury.

Several additional limitations of this analysis should be noted. Only one dose of crizanlizumab was administered, and it is possible that a higher dose at more frequent intervals may be necessary to produce significant effects. Nevertheless, the P-selectin reduction observed was substantial and sustained, suggesting that a higher dose is likely not needed. Crizanlizumab was administered relatively early in the hospital stay, and it is possible that administration even earlier in the course of COVID-19 such as the time of admission may have more impact on vascular inflammation.

In summary, crizanlizumab, a selective P-selectin inhibitor, caused a rapid, profound and sustained reduction in P-selectin levels in hospitalized patients with mild to moderate COVID-19. These findings were associated with an increase in D-dimer levels and reduction in prothrombin fragment 1.2, suggesting that crizanlizumab might increase endogenous thrombolysis in this setting. These findings raise the possibility that crizanlizumab might be clinically beneficial in patients with more severe disease.

TABLE 1
Characteristics of the Analyzed Patients at Baseline.

| Characteristic | Placebo (n = 20) | Crizanlizumab (n = 22) |
|---|---|---|
| Age | 58.0 ± 17.7 | 54.6 ± 13.4 |
| Male sex | 11 (55.0%) | 13 (59.1%) |
| Race | | |
| Asian | 1 (5.0%) | 0 (0.0%) |
| Black | 6 (30.0%) | 14 (63.6%) |
| White | 13 (65.0%) | 8 (36.4%) |
| Vital Signs | | |
| BMI | 32.7 ± 9.8 | 36.2 ± 8 .0 |
| SBP | 128.3 ± 20.8 | 135.3 ± 23.4 |
| DBP | 70.4 ± 12.6 | 74.5 ± 14.5 |
| Heart Rate | 78.0 ± 18.2 | 84.3 ± 16.7 |
| Temperature | 36.6 ± 0.5 | 36.9 ± 0.8 |
| O2% | 93.8 ± 2.9 | 93.3 ± 4.2 |
| Saturation | | |
| COVID | | |
| symptoms | | |
| Fever | 12 (60.0%) | 14 (63.6%) |
| Cough | 17 (85.0%) | 20 (90.9%) |
| Dyspnea | 17 (85.0%) | 21 (95.5%) |
| Sore Throat | 5 (25.0%) | 2 (9.1%) |
| Anosmia | 3 (15.0%) | 2 (9.1%) |
| Fatigue | 18 (90.0%) | 17 (77.3%) |
| Muscle Ache | 9 (45.0%) | 14 (63.6%) |
| WHO Status | | |
| 4 | 6 (30.0%) | 2 (9.1%) |
| 5 | 13 (65.0%) | 19 (86.4%) |
| 6 | 1 (5.0%) | 1 (4.5%) |
| Medical | | |
| History | | |
| Hx | 14 (70.0%) | 17 (77.3%) |
| Hypertension | | |
| Hx HF | 4 (20.0%) | 1 (4.5%) |
| Hx CAD | 4 (20.0%) | 0 (0.0%) |
| Hx PAD | 1 (5.0%) | 0 (0.0%) |
| Hx Stroke/TIA | 0 (0.0%) | 1 (4.5%) |
| Hx Arrhythmi | 0 | (0.0%) |
| Hx DVT/PE | 0 | (0.0%) |
| Hx Smoking | 3 | (15.0%) |
| Diabetes | 8 | (40.0%) |
| CKD (no dial) | 2 | (10.0%) |
| CKD (dialysis) | 0 | (0.0%) |
| Liver disease | 1 | (5.0%) |
| Asthma | 1 | (5.0%) |
| COPD | 3 | (15.0%) |

TABLE 2
Biomarkers of the Analyzed Patients at Baseline.

| | | Placebo | Crizanlizumab | |
|---|---|---|---|---|
| Biomarker | Analysis | (n = 20) | (n = 22) | P-value |
| P-selectin | | | | |
| (ng/mL) | | | | |
| | Mean ± SD | 34 ± 15 | 30 ± 20 | 0.40 |
| | Median [IQR] | 32 [25, 43] | 28 [18, 32] | 0.13 |
| | Geometric | 32 (26,39 ) | 25 (18,33 ) | 0.15 |
| | Mean (CI) | | | |
| IL6 (pg/mL) | | | | |
| | Median [IQR] | 71 [18, 371] | 62 [36, 87] | 0.51 |
| | Geometric | 78 (36 , 171) | 55 (33,91 ) | 0.43 |
| | Mean (CI) | | | |
| TNF -alpha | | | | |
| (pg/mL) | | | | |
| | Median [IQR] | 15 [11,22] | 13 [7, 26] | 0.34 |
| | Geometric | 16 (11,24) | 12 (7, 19) | 0.29 |
| | Mean (CI) | | | |
| VWF (lU/mL) | | | | |
| | Median [IQR] | 2.5 [1.5, 5.6] | 2.4 [1.8, 4.1] | 0.78 |
| | Geometric | 2.9 (2.0, 4.3) | 2.5 (1.9, 3.4) | 0.51 |
| | Mean (CI) | | | |
| CRP (mg/dL) | | | | |
| | Median [IQR] | 5.8 [2.7, 10.8] | 7.6 [3.2, 11.7] | 0.65 |
| | Geometric | 4.8 (2.8, 8.1) | 5.1 (3.0, 8.5) | 0.86 |
| | Mean (CI) | | | |
| D-dimer | | | | |
| (mg/L) | | | | |
| | Median [IQR] | 0.7 [0.6, 1.1] | 0.9 [0.8, 2.5] | 0.12 |
| | Geometric | 0.9 (0.6, 1.2) | 1.4 (0.9, 2.3) | 0.07 |
| | Mean (CI) | | | |

TABLE 3
Effect of Treatment on Primary Endpoint Biomarker.
Absolute change in levels of P-selectin between patients
treated with placebo and patients treated with
crizanlizumab, (95% CI) on day 3 or prior discharge.

| | Day 3 or prior | Day 7 or prior | Day 14 or prior |
|---|---|---|---|
| | discharge | discharge | discharge |
| Biomarker | [n = 42] | [n = 27] | [n =21] |
| P-selectin | −32 (−41, −24) | −38 (−48, −28) | −35 (−51, −19) |
| (ng/ml) | | | |

TABLE 4
Effect of Treatment on Primary and Secondary Endpoint
Biomarkers. Effect of crizanlizumab compared to placebo on biomarkers at day 3 or prior
to discharge, day 7 or prior to discharge, and day 14 or prior to discharge (percent change
adjusted for baseline (95% CI).

| Biomarker | Day 3 or prior discharge | Day 7 or prior discharge | Day 14 or prior discharge |
|---|---|---|---|
| P-selectin | −89% (−93%, −80%) P < 0.001 [n = 42] | −84% (−92%, −71%) P<0.001 [n = 27] | −80% (−91%, −57%) P<0.001 [n = 21] |
| IL-6 | +11% (−43%, +117%) P = 0.76 [n = 42] | −41% (−74%, +34%) P = 0.20 [n = 27] | −64% (−89%, +14%) P = 0.08 [n = 18] |
| TNF-alpha | +46% (−7%, +128%) P = 0.10 [n = 42] | +56% (−24%, +218%) P = 0.21 [n = 26] | +50% (−39%,+273%) P = 0.36 [n = 18] |
| VWF | +11% (−26%, +68%) P = 0.60 [n = 42] | +10% (−38%, +98%) P = 0.73 [n = 27] | −0% (−57%,+128%) P = 0.99 [n = 18] |
| CRP | +1% (−40%, +68%) P = 0.98 [n = 40] | −26% (−68%, +73%) P = 0.47 [n = 29] | −18% (−68%,+110%) P = 0.66 [n = 25] |
| D-dimer | +77% (+6%, +194%) P = 0.03 [n = 40] | +45% (−13%, +143%) P = 0.15 [n = 31] | +16% (−41%, +127%) P = 0.65 [n = 28] |

TABLE 5
Secondary Outcomes: Effect of treatment on clinical outcomes.
Effect of crizanlizumab (n = 22) vs. placebo (n = 20)
on clinical outcomes measured by W
Clinical Scale (median [IQR]).

| Clinical Status | Placebo (n = 20) | Crizanlizumab (n = 22) |
|---|---|---|
| WHO Status d1 | 5 [4,5] | 5 [5,5] |
| WHO Status d3 | 5 [4,5] | 5 [4,5] |

TABLE 6
Exploratory Clinical Outcomes. Effect of treatment on hospital
stay. Effect of crizanlizumab (n = 22) vs. placebo
(n = 20) on length of hospital stay
(mean ± SD).

| Days in Hospital | Placebo | Crizanlizumab | P-value |
|---|---|---|---|
| Days Admission to Randomization | 1.5 ± 0.9 | 1.7 ± 1.4 | 0.61 |
| Days Randomization to Discharge | 4.8 ± 3.5 | 6.5 ± 4.1 | 0.24 |
| Days: Admission to Discharge | 6.2 ± 3.2 | 8.1 ± 4.4 | 0.12 |

TABLE 7
Exploratory biomarkers and additional biomarkers. Absolute
levels of biomarkers of patients treated with placebo and patients treated with
crizanlizumab on baseline day 1 or on day 3 or prior discharge (n = sample size) median
[IQR]

| | Criz day 1 | Placebo day 1 | Criz day 3 | Placebo day 3 | Ratio | P- |
|---|---|---|---|---|---|---|
| Analyte | [IQR] | [IQR] | [IQR] | [IQR] | (CI) | value |
| Exploratory | | | | | | |
| biomarkers (pre- | | | | | | |
| specified) | | | | | | |
| CCL2 (pg/mL) | 257 [114, 404] | 242 [147, 293] | 243 [161, 341] | 209 [142, 460] | 0.98 (0.65, 1.49) | 0.93 |
| (n = 38) | | | | | | |
| Factor VI11 | 35 [24, 55] | 42 [26, 83] | 56 [29, 88] | 59 [32, 83] | 1.25 (0.91, 1.71) | 0.16 |
| (IU/mL) (n = | | | | | | |
| IL6 (pg/mL) | 6.3 [2.1, 10.9] | 2.1 [1.0, 10.3] | 4.8 [1.0, 12.7] | 2.1 [0.6, 5.1] | 1.14 (0.51, 2.55) | 0.75 |
| (n = 38) | | | | | | |
| TNFa (pg/mL) | 4.3 [3.3, 5.9] | 4.7 [2.8, 6.5] | 4.2 [3.4, 6.1] | 4.0 [2.7, 6.2] | 1.28 (0.95, 1.73) | 0.1 |
| (n = 38) | | | | | | |
| VCAM-1 | 24.1 [21.1, 26.6] | 24.5 [21.8, 31.0] | 23.9 [21.9, 30.2] | 23.0 [19.1, 30.0] | 1.05 (0.92, 1.21) | 0.45 |
| (ug/mL) (n | | | | | | |
| = 38) | | | | | | |
| I CAM-1 (ug/mL) | 23.4 [18.5, 26.9] | 22.6 [21.0, 25.4] | 21.0 [19.2, 27.6] | 20.7 [18.9, 23.3] | 1.08 (0.99, 1.18) | 0.1 |
| (n = 38) | | | | | | |
| hs Troponin T | 8 [3, 12] | 10 [3, 17] | 7 [3, 11] | 9 [3, 21] | 0.99 (0.72, 1.38) | 0.97 |
| (ng/mL) (n =40) | | | | | | |
| NT-proBNP | 0.19 [0.19, 0.19] | 0.19 [0.19, 0.19] | 0.19 [0.19, 0.19] | 0.19 [0.19, 0.19] | 0.99 (0.73, 1.35) | 0.97 |
| (ng/mL) (n = | | | | | | |
| Additional biomarkers | | | | | | |
| (not pre-specified) | | | | | | |
| CCL24 (pg/mL) | 195 [149, 305] | 234 [197, 301] | 182 [141, 352] | 258 [201, 351] | 0.81 (0.59, 1.11) | 0.18 |
| (n = 38) | | | | | | |
| CCL4 (pg/mL) | 92 [54, 143] | 82 [56, 116] | 128 [72, 169] | 102 [68, 136] | 1.03 (0.79, 1.34) | 0.81 |
| (n = 38) | | | | | | |
| CCL26 (pg/mL) | 20 [20, 20] | 20 [20, 20] | 20 [20, 20] | 20 [20, 20] | 1.31 (0.90, 1.91) | 0.16 |
| (n = 38) | | | | | | |
| CCL17 (pg/mL) | 103 [76, 161] | 175 [82, 257] | 103 [74, 145] | 146 [94, 240] | 0.74 (0.54, 1.02) | 0.06 |
| (n = 38) | | | | | | |
| CXCL10 | 14160 [3335, | 3935 [1820, | 5360 [2762, | 1675 [985, 5930] | 1.43 (0.86, 2.39) | 0.16 |
| (pg/mL) | 14160] | | 11125] | | | |
| CCL3 (pg/mL) | 28 [28, 28] | 28 [28, 28] | 28 [28, 28] | 28 [28, 28] | 1.18 (0.93, 1.49) | 0.16 |
| (n = 38) | | | | | | |
| CCL22 (pg/mL) | 669 [509, 963] | 832 [547, 935] | 536 [444, 654] | 630 [399, 943] | 0.93 (0.70, 1.23) | 0.6 |
| (n = 38) | | | | | | |
| CCL13(pg/mL) | 126 [76, 180] | 106 [88, 156] | 119 [86, 186] | 129 [99, 162] | 0.87 (0.68, 1.13) | 0.29 |
| (n = 38) | | | | | | |
| 2IFN-gamma | 22 [10, 108] | 21 [8, 64] | 17 [5, 43] | 7 [2, 12] | 2.10 (0.80, 5.54) | 0.13 |
| (pg/mL) (n = | | | | | | |
| IL2 (pg/mL) | 0.9 [0.9, 0.9] | 0.9 [0.9, 0.9] | 0.9 [0.9, 0.9] | 0.9 [0.9, 0.9] | 0.97 (0.76, 1.24) | 0.8 |
| (n = 38) | | | | | | |
| IL4 (pg/mL) | 0.2 [0.2, 0.2] | 0.2 [0.2, 0.2] | 0.2 [0.2, 0.2] | 0.2 [0.2, 0.2] | 1.12 (0.96, 1.31) | 0.15 |
| (n = 38) | | | | | | |
| IL8 (pg/mL) | 15.7 [9.9, 24.5] | 18.9 [15.2, 21.9] | 21.7 [15.4, 49.9] | 14.6 [11.2, 21.5] | 1.88 (1.20, 2.96) | 0,007 |
| (n = 38) | | | | | | |
| IL10 (pg/mL) | 2.9 [1.2, 5.3] | 2.0 [1.1, 3.2] | 2.8 [1.3, 6.9] | 1.0 [0.3, 1.8] | 2.64 (1.34, 5.20) | 0.007 |
| (n = 38) | | | | | | |
| IL13 (pg/mL) | 4.2 [4.2, 4.2] | 4.2 [4.2, 4.2] | 4.2 [4.2, 4.2] | 4.2 [4.2, 4.2] | 1.00 (1.00, 1.00) | 0.18 |
| (n = 38) | | | | | | |
| SAA (ug/mL) | 207 [207, 207] | 207 [207, 207] | 207 [160, 207] | 207 [33, 207] | 1.48 (0.98, 2.22) | 0.06 |
| (n = 38) | | | | | | |
| TAT complex | 6.0 [3.2, 9.9] | 5.2 [3.4, 8.8] | 4.3 [3.0, 7.8] | 8.4 [4.8, 9.8] | 0.73 (0.50, 1.05) | 0.08 |
| (ng/mL) (n = 41) | | | | | | |
| Prothrom bin | 2.6 [1.6, 4.0] | 2.5 [1.6, 3.5] | 2.3 [1.2, 3.8] | 3.6 [2.6, 6.1] | 0.50 (0.31, 0.82) | 0.007 |
| frag1.2 (ng/mL) | | | | | | |
| PAP complex | 409 [290, 960] | 397 [260, 755] | 312 [205, 737] | 345 [180, 788] | 0.98 (0.65, 1.49) | 0.93 |
| (ng/mL) (n = 42) | | | | | | |

TABLE 8
Adverse Events. Adverse events in all subjects
who received crizanlizumab or placebo.

| Adverse | Placebo | Crizanlizumab | | |
|---|---|---|---|---|
| Events | (n = 25) | (n = 25) | Total | P-value |
| Total adverse | 6 | 7 | 13 | 1 |
| events | | | | |
| Patients with any | 6 | 4 | 10 | 0.73 |
| adverse events | | | | |
| Total moderate or | 3 | 2 | 5 | 1 |
| severe adverse | | | | |
| events | | | | |
| Patients with any | 3 | 2 | 5 | 1 |
| moderate or | | | | |
| severe adverse | | | | |
| events | | | | |
| Total serious | 1 | 0 | 1 | 1 |
| adverse events | | | | |
| Patients with | 1 | 0 | 1 | 1 |
| any serious | | | | |
| adverse events | | | | |

TABLE 9
Inclusion and Exclusion Criteria

| | |
|---|---|
| Inclusion criteria | 1. Willing to provide written informed consent. |
| | 2. Willing to comply with all study procedures and be available for the |
| | duration of the study |
| | 3. Male orfemale >18 years of age |
| | 4. SARS-CoV-2 infection (COVID-19) within the past 10 d documented |
| | by laboratory test (NAT or RT-PCR) |
| | 5. Currently hospitalized |
| | 6. Symptoms of acute respiratory infection (at least one of the following: |
| | cough, fever > 37.5° C., dyspnea, sore throat, anosmia) within past |
| | 10 days |
| | 7. Radiographic evidence of pulmonary infiltrates |
| | 8. Requiring supplemental oxygen or the peripheral capillary oxygenation |
| | saturation (SpO2) < 94% on room air at screening. |
| | 9. Elevated D-Dimer > 0.49 mg/L |
| | 10. Negative pregnancy test for females of childbearing potential |
| Key Exclusion criteria | 1. Use of home oxygen at baseline |
| | 2. Current use of mechanical ventilation |
| | 3. Inability to provide informed consent |
| | 4. Do Not Intubate status |
| | 5. Prisoner or incarcerated status |
| | 6. Pregnant or nursing (lactating) women. |
| | 7. Participation in other interventional drug trials for COVID-19 |
| | (emergency use or unblinded use of convalescent plasma |
| | is permitted). |
| | 8. INR > 3 or aPTT > 60 |

TABLE 10
Sources of commercially available immunoassays used in the study.

| Supplier | Analyte |
|---|---|
| R&D Systems (Minneapolis, MN) | Soluble P-selectin |
| | IL-6 |
| | TNFalpha |
| Abeam (Cambridge, MA) | VWF |
| | Fibrinogen |
| | Thrombin-antithrombin complex |
| Lifespan Biosciences (Seattle, WA) | NT-proBNP |
| | Factor VIII |
| | Prothrombin fragment 1.2 |
| | Plasmin-antiplasmin complex |
| Roche Diagnostics (Indianapolis, IN) | Troponin T |
| Meso Scale Diagnostics (Rockville, MD) | CCL2 |
| | CCL3 |
| | CCL4 |
| | CCL13 |
| | CCL22 |
| | CCL24 |
| | CCL26 |
| | CXCL10 |
| | IL-2 |
| | IL-4 |
| | IL-8 |
| | IL-10 |
| | IL-13 |
| | Serum amyloid A |
| | VCAM-1 |
| | ICAM-1 |

The application provides the following embodiments.

1. A method of reducing the risk of thrombosis in a subject in need thereof comprising administering to the subject a therapeutically effective amount of a P-selectin inhibitor, thereby reducing the risk of thrombosis in the subject.

2. The method of embodiment 1, wherein the P-selectin inhibitor is selected from an antibody, a nucleic acid molecule or a small molecule.

3. The method of embodiment 2, wherein the antibody is crizanlizumab.

4. The method of embodiment 2, wherein the nucleic acid molecule is a short interfering nucleic acid (siNA), short interfering RNA (siRNA), double-stranded RNA (dsRNA), micro-RNA (miRNA), antagomir, aptamer, or a short hairpin RNA (shRNA) molecule.

5. The method of embodiment 4, wherein the aptamer is ARC5692.

6. The method of embodiment 2, wherein the small molecule is PSI-697.

7. The method of embodiment 1, wherein the subject with a risk of thrombosis is infected with SARS-CoV-2.

8. The method of embodiment 1, wherein the risk of thrombosis is associated with a condition selected from a microvascular, autoimmune and/or inflammatory condition.

9. The method of embodiment 8, wherein the microvascular condition is selected from microvascular thrombosis, thrombotic thrombocytopenic purpura (“TTP”), hemolytic uremic syndrome (“HUS”) or Disseminated Intravascular Coagulation (“DIC”).

10. The method of embodiment 8, wherein the autoimmune condition is selected from a type of vasculitis, rheumatoid arthritis (“RA”) or systemic lupus erythematosus (“SLE”).

11. The method of embodiment 8, wherein the inflammatory condition is selected from organ inflammation, acute transplant rejection, acute kidney allograft rejection, acute cardiac allograft rejection, chronic transplant rejection, chronic cardiac allograft rejection, or chronic renal allograft rejection.

12. The method embodiment 1, wherein the subject is at risk of acute brain injury during a procedure requiring a cardiac bypass.

13. The method of embodiment 1, wherein administration of the P-selectin inhibitor leads to a reduction of soluble P-selectin in the subject.

14. The method of embodiment 1, wherein administration of the P-selectin inhibitor leads to a reduction of prothrombin fragment 1.2 or TAT complex or thrombin generation in the subject.

15. The method of embodiment 1, wherein the P-selectin inhibitor is administered to the subject as a pharmaceutical composition in a therapeutically effective amount.

16. The method of embodiment 15, wherein the pharmaceutical composition is administered via an enteral, topical or parenteral route of administration.

17. The method of embodiment 16, wherein the route of administration is selected from intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, or administration via infusion, inhalation, or nebulization.

18. The method of embodiment 15, wherein the P-selectin inhibitor is administered via an intravenous route.

19. The method of embodiment 18, wherein the P-selectin inhibitor is administered to the subject at a dosage of about 5 mg/kg.

20. The method of embodiment 19, wherein the P-selectin inhibitor is administered to the subject as a single intravenous infusion.

21. A method of treating a SARS-CoV2 associated endothelial injury in a subject comprising administering to the subject a P-selectin inhibitor and/or a VWF inhibitor, thereby treating the SARS-CoV2 associated endothelial injury.

22. The method of embodiment 21, wherein the P-selectin inhibitor is crizanlizumab, PSI-697 or ARC5692.

23. The method of embodiment 21, wherein the VWF inhibitor is integrilin or abciximab.

24. The method of embodiment 21, wherein the P-selectin inhibitor and/or the VWF inhibitor decrease vascular inflammation and vascular thrombosis.

25. The method of embodiment 21, wherein the P-selectin inhibitor and/or the VWF inhibitor inhibit endothelial-leukocyte interaction and endothelial-platelet interaction.

26. The method of embodiment 20, wherein the infusion is administered once a week, once every 2 weeks, once every three weeks, once every four weeks, once every five weeks, once every six weeks, once every seven weeks, or once every eight weeks.

Although the invention has been described with reference to the above examples, it will be understood that modifications and variations are encompassed within the spirit and scope of the invention. Accordingly, the invention is limited only by the following claims.

What is claimed:

1. A method of reducing the risk of thrombosis in a subject in need thereof comprising administering to the subject a therapeutically effective amount of a P-selectin inhibitor, thereby reducing the risk of thrombosis in the subject.

2. The method of claim 1, wherein the P-selectin inhibitor is selected from an antibody, a nucleic acid molecule or a small molecule.

3. The method of claim 2, wherein the antibody is crizanlizumab.

4. The method of claim 2, wherein the nucleic acid molecule is a short interfering nucleic acid (siNA), short interfering RNA (siRNA), double-stranded RNA (dsRNA), micro-RNA (miRNA), antagomir, aptamer, or a short hairpin RNA (shRNA) molecule.

5. The method of claim 4, wherein the aptamer is ARC5692.

6. The method of claim 2, wherein the small molecule is PSI-697.

7. The method of claim 1, wherein the subject with a risk of thrombosis is infected with SARS-CoV-2.

8. The method of claim 1, wherein the risk of thrombosis is associated with a condition selected from a microvascular, autoimmune and/or inflammatory condition.

9. The method of claim 8, wherein the microvascular condition is selected from microvascular thrombosis, thrombotic thrombocytopenic purpura (“TTP”), hemolytic uremic syndrome (“HUS”) or Disseminated Intravascular Coagulation (“DIC”).

10. The method of claim 8, wherein the autoimmune condition is selected from a type of vasculitis, rheumatoid arthritis (“RA”) or systemic lupus erythematosus (“SLE”).

11. The method of claim 8, wherein the inflammatory condition is selected from organ inflammation, acute transplant rejection, acute kidney allograft rejection, acute cardiac allograft rejection, chronic transplant rejection, chronic cardiac allograft rejection, or chronic renal allograft rejection.

12. The method of claim 1, wherein the P-selectin inhibitor is administered to the subject as a pharmaceutical composition in a therapeutically effective amount.

13. The method of claim 12, wherein the pharmaceutical composition is administered via an enteral, topical or parenteral route of administration.

14. The method of claim 16, wherein the route of administration is selected from intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, or administration via infusion, inhalation, or nebulization.

15. The method of claim 14, wherein the P-selectin inhibitor is administered to the subject by intravenous infusion at a dosage of about 5 mg/kg.

16. The method of claim 15, wherein the P-selectin inhibitor is administered to the subject as a single intravenous infusion.

17. The method of claim 1, further comprising administering a VWF inhibitor.

18. The method of claim 17, wherein the P-selectin inhibitor is crizanlizumab, PSI-697 or ARC5692.

19. The method of claim 18, wherein the VWF inhibitor is integrilin or abciximab. 4.

20. The method of claim 1, wherein the infusion is administered once a week, once every 2 weeks, once every three weeks, once every four weeks, once every five weeks, once every six weeks, once every seven weeks, or once every eight weeks.
